(19) United States
(12) Patent Application Publication
(10) Pub. No.: US 2026/0027149 A1
(43) Pub. Date: Jan. 29, 2026

(54) NITRIC OXIDE GAS-INFUSED LIQUIDS AND RELATED METHODS

(71) Applicant: ThermoLife International, LLC, Signal Hill, CA (US)

(72) Inventors: Ronald KRAMER, Signal Hill, CA (US); Alexandros NIKOLAIDIS, Nea Kallikratia (GR)

(21) Appl. No.: 19/283,219

(22) Filed: Jul. 28, 2025

Related U.S. Application Data
(60) Provisional application No. 63/676,198, filed on Jul. 26, 2024.
(60) Provisional application No. 63/797,026, filed on Apr. 29, 2025.

(51) Int. Cl. A61K 33/00; A61K 9/00; A61P 7/00; C01B 21/24

(52) U.S. Cl. CPC A61K 33/00; A61K 9/0078; A61P 7/00; C01B 21/24

(57) ABSTRACT

A novel method of producing an NO+H₂ solution is disclosed as well as liquid compositions comprising stable and high concentrations of NO and H₂.

### RELATED APPLICATIONS

This application claims priority to and the benefit of U.S. provisional patent 63/676,198, filed Jul. 26, 2024 to Ronald Kramer et al., titled “Nitric Oxide Gas-Infused Liquids and Related Methods” and also claims priority to and the benefit of U.S. provisional patent 63/797,026, filed Apr. 29, 2025 to Ronald Kramer et al., titled “Nitric Oxide Gas-Infused Liquids and Related Methods,” the entirety of each of the disclosures of which are hereby incorporated by reference herein.

### TECHNICAL FIELD

The disclosure relates a method of infusing nitric oxide gas into a solution that overcomes the low stability of the gas in water as well as other aqueous solutions and liquids.

### BACKGROUND

The solubility of nitric oxide gas (NO) in water has been reported to be 0.0098 g/100 ml in 0° C., 0.0056 g/100 ml in 20° C., or 1.94±0.03×10⁻⁶ mol/cm³/atm.

Nitric oxide (NO) is rapidly converted to NO₂ by oxidation with oxygen in the atmosphere, so NO is notoriously unstable in water and other aqueous solutions and liquids (such as plasma and blood). It has been taught that the preparation of saturated NO solutions involves “meticulous exclusion of O₂, as NO is rapidly destroyed by O₂” (Archer, “Measurement of nitric oxide in biological models,” FASEB J, 1993; 7(2):349-60, hereinafter “Archer”). Archer also notes that even if the NO solution is hermitically sealed in a glass ampule in a N₂ atmosphere, NO is only stable for “several days”. In water, NO is in contact with the oxygen dissolved in water as well as the atmospheric oxygen at surface level, so NO₂ is formed which then may further react with water to form HNO₃ (nitric acid).

The half-life of NO in water once in contact with atmosphere has been described to be a mere 445 seconds, and is purported to be eliminated in blood in under 2 minutes. To the best of anyone's knowledge, no appreciable amount of NO can be found in the blood or other bodily fluids, as the gas is rapidly oxidized and/or reacts with other molecules to form compounds like S-nitrosothiols (RSNO) and nitrosyl-hemoglobin. Thus, producing stable NO solutions for experimentation or other uses has been problematic.

The usual method of infusing a liquid with NO to produce NO solutions starts with producing a purified gas mixture which is diffused to water. The purified gas mixture is produced by passing a mixture containing NO (such as an NO/NO₂ mixture formed by reaction of sodium nitrite with an acid, such as sulfuric acid) or NO from a gas canister (where usually it is kept stable by addition of N₂) through a sodium hydroxide solution deoxygenated with argon or another gas like helium (both of them expensive) to remove NO₂/HNO₃. The whole line and solutions have to be purified by argon or another inert gas capable of displacing oxygen over a long time and the whole system must be kept hermitically sealed to prevent intrusion of oxygen. Experienced artisans in the field find it challenging to prevent oxygen intrusion during this process, even in a small scale set-up. Other conventional methods to remove oxygen are incapable of removing all the oxygen from the solution.

Many methods are known for diffusing gases into a liquid, and they generally comprise passing the gases through the liquid, preferably from the bottom. Other options can exist that would diffuse the gases in the liquid, such as using a press to push the gases into the liquid. Pipes, tubes, syphones, diffusion stones, funnels, and similar apparatus capable of passing the gas through the liquid can be used.

After the NO solution is made, it must be stored in hermitically closed containers making sure no oxygen is present (see Archer).

A further obstacle of an NO solution's suitability for administering NO is limited by the fact that the limited NO presented in the administered liquid likely rapidly disappears in the environment of the mouth and the stomach, where both atmospheric and dissolved oxygen are abundantly present, and is unavailable for effecting any physiological change.

Thus, unless other methods of creating NO solutions are developed, including those stable in the presence of oxygen, solutions comprising dissolved NO are not expected to be a practical, economical, or suitable route of administering NO to a subject or to provide any benefits of NO.

### SUMMARY

Disclosed herein is a composition comprising a liquid, nitric oxide gas (NO), and hydrogen gas (H₂), wherein the NO and the H₂ are diffused in the liquid. In some aspects, the NO and the H₂ in the composition are dissolved in the liquid. In some implementations, the liquid in the composition is water or comprises water. In some implementations, the concentration of the NO and the H₂ in the composition are respectively at least 1 ppm and at least 100 ppb.

In certain implementations, the composition further comprises a pharmaceutically acceptable additive. In some aspects, the pharmaceutically acceptable additive is selected from: a carrier, excipient, binder, colorant, flavoring agent, preservative, buffer, diluent, and combinations thereof. In particular implementations, the pharmaceutically acceptable additive is a food additive.

Disclosed also herein is a nitric oxide and hydrogen (NOH2) administration container that includes at least: a liquid; nitric oxide gas (NO); and hydrogen gas (H2). The NO and the H₂ are diffused in the liquid to produce a nitric oxide stabilized liquid.

Implementations may include one or more or all of the following.

The NO and the H₂ may be dissolved in the liquid either sequentially or at the same time.

The liquid may include water.

Concentration of the NO and the H₂ may be respectively at least 1 ppm and at least 100 ppb.

A pharmaceutically acceptable additive or additive may be included. The additive or pharmaceutically acceptable additive may be selected from the group consisting of: a carrier, an excipient, a binder, a colorant, a flavoring agent, a preservative, a buffer, a diluent, and/or combinations thereof. The additive or pharmaceutically acceptable additive may be a botanical additive derived from a plant including a fruit of a plant. The additive or pharmaceutically acceptable additive may be a food additive. The food additive may be a liquid, such as milk for example.

The liquid may include blood.

The liquid may include a botanical liquid. The botanical liquid may be a fruit juice.

The administration container may be a beverage container having a container wall with a consumption opening and an opening closure that seals the nitric oxide stabilized liquid in the container. The beverage container may be pressurized up to 1 atmosphere or above 1 atmosphere or more. The beverage container may be pressurized with NO. The beverage container may be a metal can. The metal can may be an aluminum can.

The administration container may be an intravenous (IV) container or syringe.

The administration container may be a nebulizer container configured for use with a nebulizer or vape container configured for insertion into a vape device for inhalation of the nitric oxide stabilized liquid as a vapor therefrom.

The administration container may be capsule for oral consumption.

Disclosed is a method of producing a nitric oxide stabilized liquid having stabilized nitric oxide gas (NO) dissolved therein (or a method of reducing degradation of NO concentration in a liquid). The method includes: providing a nitric oxide liquid with NO dissolved therein with a concentration of least 1 ppm; providing hydrogen (H2); and adding or dissolving the H₂ in the liquid at a concentration of at least 100 ppb to produce the nitric oxide stabilized liquid. Alternatively, NO and H₂ can be diffused at or near the same time.

Implementations may include one or more or all of the following.

The nitric oxide stabilized liquid may include oxygen.

The NO concentration in the liquid may be stable having less than a 25% drop in NO concentration in 24 hours after the addition of H₂.

Providing a nitric oxide liquid with NO dissolved therein with a concentration of least 1 ppm may include: providing NO; providing a liquid; and dissolving the NO in the liquid to produce the nitric oxide liquid with a concentration of least 1 ppm.

Additionally disclosed is a method of producing a nitric oxide stabilized liquid. The method includes: providing a liquid; providing NO; providing H₂; and simultaneously dissolving in the liquid the NO and the H₂ to produce the nitric oxide stabilized liquid.

Implementations may include one or more or all of the following.

The NO is at a concentration of least 1 ppm and the H₂ is at a concentration of at least 100 ppb in the nitric oxide stabilized liquid.

The nitric oxide stabilized liquid may include oxygen.

The NO concentration in the liquid may be stable having less than a 25% drop in NO concentration in 24 hours after the simultaneous addition of NO and H₂.

A method of slowing spoilage of a beverage is additionally disclosed herein. The method comprises diffusing nitric oxide gas (NO) and hydrogen gas (H₂) in the beverage. In some aspects, diffusion of NO and H₂ in the beverage results in 10% lower (or even lower) bacterial and fungal growth compared to untreated beverage stored under similar conditions.

Further disclosed herein are methods of reducing or treating a disease or disorder in a subject, such as methemoglobinemia in a subject in need thereof; hypertension in a subject in need thereof, atherosclerosis in a subject in need thereof; varicose veins in a subject in need thereof, low oxygen saturation/hypoxia in a subject in need thereof, gastrointestinal ulcers in a subject in need thereof, viral, fungal, bacterial, protozoa, or parasitic infections in a subject in need thereof, migraine in a subject in need thereof, anemia in a subject in need thereof, inflammation in a subject in need thereof, high F2-isoprostane levels in a subject in need thereof; and apnea/difficulty breathing in a subject in need thereof. The methods of reducing or treating a disease or disorder in a subject each my comprise administering to the subject a pharmaceutically effective amount of a composition comprising water, nitric oxide gas (NO), and hydrogen gas (H₂), wherein the NO and the H₂ are at concentrations of least 1 ppm and 100 ppb respectively.

In some implementations of the methods of treating a disease or disorder in a subject, the composition comprising NO and H₂ at concentrations of least 1 ppm and 100 ppb respectively is administered orally in a dose of about 1-10,000 ml. In certain implementations, the composition is administered orally in a dose of about 10-5,000 ml. In particular implementations, the composition is administered orally in a dose of about 100-1,000 ml and any other range between and including the dose values provided.

In other implementations of the methods of treating a disease or disorder in a subject, the composition is administered intravenously. For example, the composition is administered at an infusion rate of about 0.1-10 ml/min.

Still further disclosed are methods of increasing nitric oxide levels in blood of a subject; increasing plasma H₂ level in a subject; and reducing plasma and/or muscle lactate levels produced during exercise in a subject. These methods comprise orally administering to the subject the composition described.

In some implementations of the method of increasing nitric oxide levels in a subject, the subject is orally administered at least 100 ml of a composition prepared according to a method comprising: combining an effective dose of potassium Nitrate (KNO₃)(such as 10 g KNO₃), an effective dose of magnesium metal powder (such as 2.5 g magnesium metal powder (60-200 mesh size)), and an effective dose of citric acid (such as 19 g of citric acid) to produce a gaseous composition comprising nitric oxide gas (NO) and hydrogen gas (H₂); and diffusing 500 ml water with the produced gaseous composition at 0° C. for 5 minutes. In some aspects, the increased nitric oxide level is measured by reaction of blood plasma taken from the subject 0.1 to 24 hours after oral administration of the composition.

In some implementations of the method of increasing plasma H₂ level in a subject; the subject is orally administered the composition comprising NO and H₂, and the concentration of H₂ in the plasma is measured by reacting plasma with methylene blue in the presence of a platinum catalyst, or with other suitable methods. In some aspects, the increase in H₂ levels is at least 10% greater than that of drinking water or another liquid that contains a similar amount of H₂ but no NO. In some implementations, the increase in plasma NO levels in measured by reacting plasma with ABTS (2,2′-azinobis(3-ethylbenzothiazoline-6-sulfonic acid) or another suitable reactant.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The accompanying drawings are incorporated in and constitute a part of this specification, illustrate and provide a further understanding of implementations of the present disclosure, and together with the specification serve to explain the principles of the present disclosure.

FIG. 1 shows an example of an infusion apparatus for infusing nitric oxide gas (NO) into water, an aqueous solution, or other liquid, that may be used in accordance with the present disclosure.

FIG. 2 shows a nitric oxide and hydrogen (NOH2) administration container, such as a beverage container, with nitric oxide stabilized liquid therein.

FIG. 3 shows a capsule containing nitric oxide stabilized liquid therein.

FIG. 4 shows an intravenous (IV) container containing nitric oxide stabilized liquid therein.

FIG. 5 shows a vape container containing nitric oxide stabilized liquid therein.

FIG. 6 shows a schematic of nitric oxide inducing methemoglobinemia in blood.

FIG. 7 shows a test specimen of heparinized bovine blood with and without NO and hydrogen gas (H₂) treatment as described in Example 4.

FIG. 8 shows blood specimens with and without NO treatment as described in Example 6.

FIG. 9 shows beet juice specimens with and without NO and H₂ treatment as described in Example 9.

FIG. 10 shows a ultraviolet-visible (“UV-Vis”) spectra from a Vernier UV-Vis spectrophotometer as described in Example 15.

FIG. 11 shows a UV-Vis spectra from a Vernier UV-Vis spectrophotometer as described in Example 15.

FIG. 12 shows a UV-Vis spectra from a Vernier UV-Vis spectrophotometer as described in Example 15.

FIG. 13 shows a UV-Vis spectra from a Vernier UV-Vis spectrophotometer as described in Example 15.

FIG. 14 shows a UV-Vis spectra from a Vernier UV-Vis spectrophotometer as described in Example 15.

FIG. 15 shows a UV-Vis spectra from a Vernier UV-Vis spectrophotometer as described in Example 15.

FIG. 16 shows a wavelength-absorbance graph as described in Example 15.

FIG. 17 shows a UV-Vis spectra from a Vernier UV-Vis spectrophotometer as described in Example 16.

Corresponding reference characters indicate corresponding parts throughout the several views of the figures. The figures represent an illustration of some of the implementations of the present invention and are not to be construed as limiting the scope of the invention in any manner. Some of the figures may not show all of the features and components of the invention for ease of illustration, but it is to be understood that where possible, features and components from one figure may be included in the other figures. Further, the figures are not necessarily to scale, some features may be exaggerated to show details of particular components. Therefore, specific structural and functional details disclosed herein are not to be interpreted as limiting, but merely as a representative basis for teaching one skilled in the art to employ the present invention.

### DETAILED DESCRIPTION

Detailed aspects and applications of the disclosure are further described below, in any pictures/drawings/structures, and in the claims. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant art, that the present disclosure may be practiced without these specific details. It should be noted that there are many different and alternative configurations, devices, systems, methods, and technologies to which the divulged disclosures may be applied. The full scope of the disclosures is not limited to the examples that are described below.

The singular forms “a,” “an,” and “the” include plural referents unless the context clearly dictates otherwise. Thus, for example, reference to “a step” includes reference to one or more of such steps.

“About” as used herein when referring to a measurable value such as an amount, a temporal duration, and the like, is meant to encompass variations of 20%, from the specified value, as such variations are appropriate. It is to be understood that in the present specification, the use of the term “about” in connection with a numerical value also affords support for the exact numerical value as though it had been recited without the term “about” and for any number within the ±20% range.

As used in herein, the term “pharmaceutically acceptable” is used in its broadest sense and may describe the quality of meeting Food and Drug Administration (FDA) standards, United States Pharmacopeial Standards (USP), US Department of Agriculture (USDA) standards for food-grade materials, commonly accepted standards of the nutritional supplement industry, industry standards, or botanical standards. These standards may delineate acceptable ranges of aspects of ingredients of a pharmaceutical composition such as edibility, toxicity, pharmacological effect, or any other aspect of a chemical, composition, or preparation used in implementations of a pharmaceutical composition.

As used herein, the term “disease” refers to a state of health of an animal (including humans) wherein the animal cannot maintain homeostasis, and wherein if the disease is not ameliorated then the animal's health continues to deteriorate.

In contrast, as used herein, the term “disorder” in an animal refers to a state of health in which the animal (including humans) is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

A disease is “cured” if the disease no longer shows any symptoms and appropriate diagnostic tests show absence of it on a patient.

As used herein, “treat,” “treatment,” and “treating,” a disease or disorder means reducing the severity and/or frequency, with which a sign and/or symptom of the disease or disorder is experienced by a subject. The subject may be symptomatic or asymptomatic at the time of treatment. In other words, “treat,” “treatment,” and “treating,” can be to reduce, ameliorate or eliminate signs or symptoms associated with a condition present in a subject, or can be metaphylactic or prophylactic, (i.e. to prevent or reduce the occurrence of the symptoms in a subject, or to delay onset of possible or expected signs or symptoms in a subject). Such prophylactic or metaphylactic treatment can also be referred to as prevention of the condition.

As used herein, “pharmaceutically acceptable additive” or “additive” are terms used in their broadest sense. Particular implementations of the compositions described in this document may also comprise an additive (e.g. one of a solubilizer, an enzyme inhibiting agent, an anticoagulant, an antifoaming agent, an antioxidant, a coloring agent, a coolant, a cryoprotectant, dilutant, a hydrogen bonding agent, a flavoring agent, a flow agent, a plasticizer, a preservative, a sweetener, a thickener, and combinations thereof) and/or a carrier (e.g. one of an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof). These additives may be solids or liquids, and the type of additive may be generally chosen based on the type of administration being used. Those of ordinary skill in the art will be able to readily select suitable pharmaceutically acceptable additives from the disclosure in this document. In particular implementations, pharmaceutically acceptable additives may include, by non-limiting example, calcium phosphate, cellulose, stearic acid, crosscarmelose cellulose, magnesium stearate, and silicon dioxide.

As used in this document, “pharmaceutically effective” is a phrase used in its broadest sense, including, by non-limiting example, effective in a clinical trial or for a specific patient. When used in a method claim, pharmaceutically effective will mean in a dose enough to achieve the claim's preamble.

As used herein, the term “effective amount” or “therapeutically effective amount” of a compound is that amount of compound which is sufficient to accomplish a specified task or function desired of the compound, for example, to provide a beneficial effect to the subject to which the compound is administered.

The term “pharmaceutically effective amount,” as used herein, refers to an amount that is sufficient or effective to prevent or treat (delay or prevent the onset of, prevent the progression of inhibit, decrease or reverse) a disease or disorder in a subject or produce any other beneficial physiological changes to the subject due to changes in the subject's homeostasis, such as increase in athletic performance and mood. It is understood that various biological factors may affect the ability of a substance to perform its intended task. Therefore, the pharmaceutically effective amount may be dependent in some instances on such biological factors.

Turning to other aspects of this disclosure, described herein is a novel method of infusing a liquid, such as water, blood, or juice, with nitric oxide gas (NO) and hydrogen gas (H₂) (in amounts greater than those naturally found in water) to produce an NO+H₂ solution that does not require the need to purge oxygen from the liquid or atmosphere is described herein. The NO+H₂ solutions produced from the novel method have stable concentrations of NO for extended periods of time, even in the presence of ambient heat or sunlight. The NO+H₂ solutions also commonly remain at a neutral or slightly alkaline pH in spite of NO₂ formed by reaction of NO with oxygen being known to form nitric acid (HNO₃). Furthermore, the NO+H₂ solutions are relatively cheap and easy to make according to the novel method disclosed herein.

As used herein, the term “NOH2 water” refers to a NO+H₂ solution that may be produced according to the novel method disclosed herein where the solvent is water. It will be understood that for aqueous solutions containing other solvents or solutions of dissolved substances or solids such as juices, blood products, the term “NOH2” is a modifier identifying such solutions as comprising NO and H₂ that may be produced according to the method described herein. Accordingly, if blood is infused with NO and H₂ it will be referred to herein as “NOH2 blood”, while orange juice infused with NO and H₂ will be referred to herein as “NOH2 orange juice.” In other words, liquids with the modifier “NOH2” shall mean the respective liquids comprise NO and H₂ concentrations much higher than those found in nature at standard atmospheric conditions, standard atmospheric pressure and at standard room temperature of 22° C. For example, human blood typically contains 0.36 ng/L or 0.36 parts per trillion (ppt) of NO at said standard atmospheric conditions. For example, in the case of water, water from natural sources (whether rainwater, spring water, ocean water, condensation, and the like) contains no NO dissolved within.

In general, the method of infusing a liquid with NO to produce an NO+H₂ solution comprises diffusing H₂ and NO through a liquid that can be near-freezing temperature to allow for greater diffusion (but still in full or part liquid state), for example at around 0° C. for water or a potentially lower temperature for a water-based solution (for example, about −1 to −2° C. for 2-3% saline solution or certain juice, about −2 to −3° C. for blood, or about −0.5 to −0.6° C. for plasma). The sources of NO can be any appropriate NO-producing or storing device or apparatus, such as a gas tank or a chemical composition that produces NO. One such novel exemplary chemical composition that produces NO, developed by the inventors, is a combination of a source of nitrate anion and/or nitrite anion, the uncharged form of a metal (also referred to herein as “an elemental metal”, for example the uncharged form of magnesium may be referred to as elemental magnesium metal), and an acid. This novel method can produce no harmful gases such as NH3, NO2 and N2O and as an added advantage also simultaneously produces hydrogen. Hydrogen can also be provided by any appropriate H₂ producing or storing device or apparatus, such as a hydrogen gas tank or an elemental (uncharged) metal reacting with an acid or a metal hydride in contact with water.

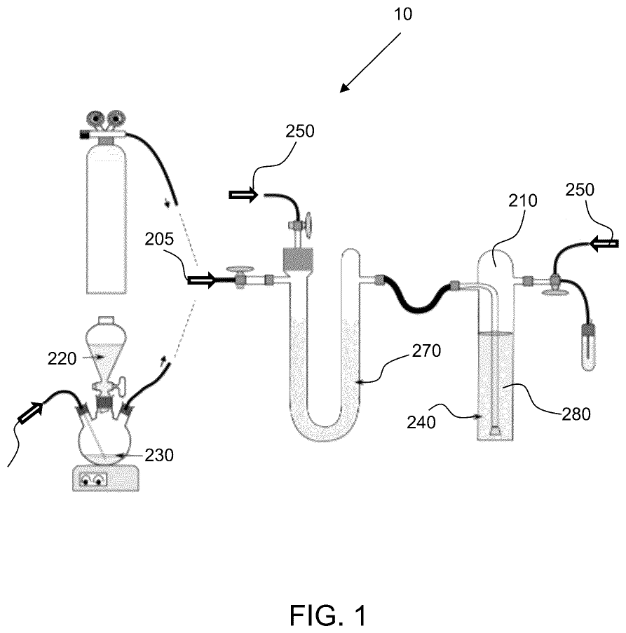

As shown in FIG. 1, infusing a liquid with NO 205 to produce NO solutions starts with producing a purified gas mixture 210. The purified gas mixture is produced by passing a mixture containing NO (such as an NO/NO₂ mixture formed by reaction of sodium nitrite 220 with an acid 230, such as sulfuric acid) or NO from a gas canister (where usually it is kept stable by addition of N₂) through a sodium hydroxide solution 240 deoxygenated with argon 250 or another gas like helium (both of them expensive) to remove NO₂/HNO₃. NaOH 270 pellets may be configured in the flow of NO/NO₂ flow to the infuser vessel 280. The whole line and solutions have to be purified by argon or another inert gas capable of displacing oxygen over a long time and the whole system must be kept hermitically sealed to prevent intrusion of oxygen.

In certain implementations, H₂ and NO are diffused in or delivered to the liquid from the same vessel and through the same tubing. The chemical method of producing NO involving a source of nitrate anion or nitrite anion and an elemental metal has the advantages of co-creation of NO and H₂ simultaneously, ensuring NO is not converted by NO₂ in the air and easily avoiding use of potentially unsafe chemicals with environmental concerns such as nitric acid, and also enabling easier simultaneous diffusion. Also, no notable amounts of harmful gases such as NH3, N2O and NO2 are produced but these by-products can be removed or reduced to enable uses described herein. Thus, in certain implementations, H₂ and NO are produced from combining a source of nitrate anion with an elemental metal in an acidic solution. The gases produced from the reaction are delivered to the liquid to facilitate infusion of the liquid with H₂ and NO. In other implementations, the method of infusing a liquid with NO to produce an NO+H₂ solution comprises mixing an elemental metal, an acid, and source of nitrate anion and/or nitrite anion with the liquid while keeping the temperature low, for example near freezing point, and for example in a closed container. Because the solution comprises byproducts of the metal, acid, and the source of nitrate anion and/or nitrite anion, the resulting NO+H₂ solution may not be suitable for all of the described uses of the NO+H₂ solution.

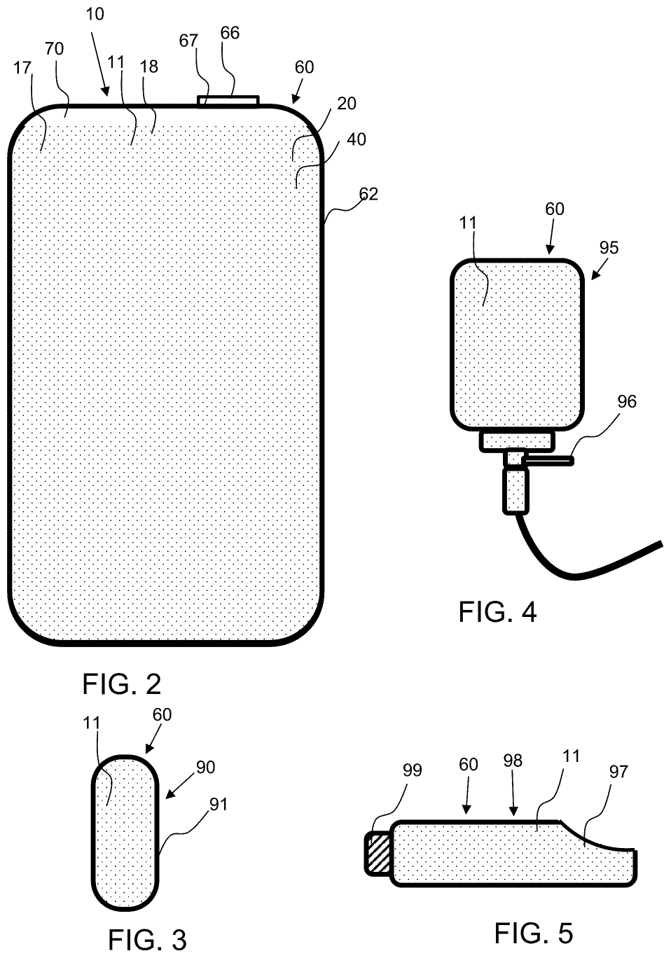

As shown in FIG. 2, the nitric oxide stabilized liquid 11, includes a liquid 17, such as water 18, to form NOH2 water 11, with a concentration of NO 40 and H₂ 20. The nitric oxide stabilized liquid 11 is contained in a nitric oxide and hydrogen (NOH₂) administration container 60, as described herein, having a container wall 62 that may retain the nitric oxide stabilized liquid 11 under elevated pressure (e.g., more than about one atmosphere, up to about three or more atmosphere, four or more atmosphere, five or more atmosphere, and even ten or more atmosphere, and any range between, and including the pressure values provided). The NOH2 administration container 60 may be a beverage container 10 and have a consumption opening 67 with a container closure 66 to seal the consumption opening. Also, the NOH2 administration container, such as beverage container 10, may be pressurized with another gas, such as carbon dioxide, that may also be in a head space 70 of the container. The beverage container 10 may be pressurized with NO and/or a combination of NO and H₂ and in some cases also carbon dioxide (all of which may also be in a head space, e.g., 70, of the container). The beverage container may prevent diffusion of gasses through the container wall, and the container wall may be metal, such as aluminum, or lined aluminum.

The NOH2 administration container 60 may be a capsule 90 with a wall 91 that contains the nitric oxide stabilized liquid 11 as shown in FIG. 3.

The NOH2 administration container 60 may be an IV container 95 such as an IV bag that contains the nitric oxide stabilized liquid 11 as shown in FIG. 4, having a flow regulator 96.

The NOH2 administration container 60 may be a vape container 98 for use with a vaping device to inhale the nitric oxide stabilized liquid 11 as shown in FIG. 5 and may have a vape pen interface 99, such as a threaded coupling, and may have an oral interface 97 for inhaling therefrom.

For the exemplary purposes of this disclosure, the concentration of NO in an NO-containing gas mixture to be infused is between 0.1 to 1,000,000 ppm, more specifically between 1-100 ppm, and even more specifically between 5-40 ppm. Such a concentration ensures that minimal, if any, conversion of NO to NO₂ takes place, even in the air before the gases are mixed in the liquid.

For the exemplary purposes of this disclosure, the concentration of H₂ in the NO+H₂ solution is between 0.01-1,000 ppm, more specifically between 0.1-100 ppm, and even more specifically between 0.1-10 ppm. This is an increase from typical concentrations of H₂ that may be dissolved in water. Regular drinking water contains insignificant amounts of H₂, about 0.8 ppt (parts per trillion) or less.

The aforementioned concentrations of NO and H₂ ensure only a minimal amount of undissolved NO and H₂ or other potentially harmful gases such as NO₂, N₂O and NH₃ escape to the atmosphere, which minimizes occupational hazards related to the production of the NO+H₂

### Solution

The liquids in which NO and H₂ are infused according to the described method include water or any other mixture of water with other liquids or solids or any other liquid where NO and H₂ are substantially soluble. For example, hypertonic, isotonic or hypotonic solutions, such as saline 0.9%, ringers, or 5% dextrose could be used for the production of a solution for intravenous administration. Blood products such as whole blood, plasma, or platelet rich plasma could be used for intravenous infusion too. In the case of orally ingested solutions, water as well as any liquid suitable for oral ingestion, such as juices, protein shakes, syrups, tinctures, suspensions, elixirs and concentrates could be used. Furthermore, other liquid substances, preferably miscible with water, such as glycerol or propionyl glycol, may be used instead of or in conjunction with water.

Solubility and content of NO in the solutions can be detected by various methods, such as chemiluminescence, oxidation with an oxidant such as oxygen to nitrite/nitrate and subsequent determination of the concentration of the ions via, for example, the Griess reaction, or colorimetry (UV-Vis) with 2,2′-Azino-bis(3-ethylbenzothiazoline-6-sulfonic acid).

Hydrogen can also be determined by known methods, such as use of a direct in water dissolved hydrogen meter, a hydrogen sensor, GC-MS, methylene blue in conjunction with colloidal platinum reactants, and the like.

Also described herein is a more convenient method for approximating both hydrogen and nitric oxide in a solution. The method comprises enclosing the solution in an (optionally) heated airtight chamber with an appropriate NO and an H₂ electrochemical sensor. Temperature of the solution may then be increased, for example to 60° C. As the temperature of the solution rises, gas solubility reduces, and the concentration of gases in the chamber is increased. The gas concentration can easily be determined by specific gas sensors, or more complicated methods such as GC-MS. Thus, by using fixed volumes of liquid, in fixed sized chambers at fixed volume in airtight enclosures, one can approximate the amount of NO and H₂ in a solution.

A composition comprising a liquid, NO, and H₂, wherein the gases have been diffused in the liquid (for example, dissolved in the liquid) is also described. The liquid may be water or any other aqueous solution. The liquid can also be a juice from a vegetable or fruit or blood, including blood derivatives such as plasma or platelet rich plasma. The composition comprises NO and H₂ at concentrations much greater than those that can be found in the naturally occurring liquids. For example, no amount of NO is found naturally in water. In blood of subjects not under any condition or medication that might drastically alter NO concentration (such as i.v. sodium nitroprusside therapy), the typical concentration of NO is 0.36 ng/L or 0.36 parts per trillion. A suitable concentration of NO to be infused is between 0.1 to 1,000,000 ppm, more specifically between 1-100 ppm and even more specifically 5-40 ppm for example. A suitable concentration of H₂ in the NO+H₂ solution is between 0.01-1,000 ppm, more specifically between 0.1-100 ppm, even more specifically between 0.1- and 10 ppm for example.

For NOH2 water, it can be administered to provide NO and/or H₂ where administration of NO or H₂ would be beneficial. Depending on the route of administration, the dosage of NOH2 water may be varied. For oral route of administration, the dosage is between 1-10,000 ml, preferably between 10-5,000 ml, and more preferably between 100-1,000 ml. For intravenous infusion, the rate of infusion may typically be between 0.1-10 ml/min depending on the severity of disease and symptoms although higher rates of infusion can be appropriate in some instances. For inhaled administration, the dosage may be that sufficient to alleviate the symptoms. A skilled artisan can adjust the rate of infusion to ensure an appropriate dosage is administered. For dermal applications, the subject may apply the solution on his or her skin as needed (for example, washing with the NOH2 water, administered via gauzes infused with the solution, or topically applied with carriers such as jellification agents to ensure long-term contact with the skin).

In other aspects, the composition further comprises a suitable pharmaceutically acceptable coating to prevent moisture from getting in the tablets or capsules and/or an additive. Non-limiting examples of the pharmaceutically acceptable coatings include waxes, polymers, and solid fatty acids. In other aspects, the composition further comprises a suitable additive. Non-limiting examples of such an additive include a carrier, excipient, binder, colorant, flavoring agent, preservative, buffer, diluent, and combinations thereof. In some aspects, the additive is a pharmaceutically acceptable additive or an acceptable food additive. In certain implementations, the suitable additive is selected from a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, a thickener, and combinations thereof. In certain implementations, the suitable carrier includes an excipient, a lubricant, a binder, a disintegrator, a diluent, an extender, a solvent, a suspending agent, a dissolution aid, an isotonization agent, a buffering agent, a soothing agent, an amphipathic lipid delivery system, and combinations thereof.

In some implementations, the composition is contained in a sealed mono-dose liquid container. Examples of such containers include but are not limited to ampules, bottles, capsules, gels, vape cartridge, vials, cans, and jars, which are suitable for storage and carrying of the disclosed compositions. The sealed mono-dose liquid container may be a liquid or gel capsule or a transdermal patch.

In some implementations, the liquid in the composition is a foodstuff, such as juice, jam, or a drink. In such implementations, the presence of NO and H₂ may provide additional health benefits to the foodstuff or may enhance the shelf life of the foodstuff (for example, by slowing down the rate of spoilage). In some aspects, such compositions further comprise an additive and/or a nutrient, for example, vitamins, sweeteners, functional ingredients including, but not limited to, saponins, antioxidants, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols and combinations thereof.

Also disclosed herein are methods of 1) treating and/or preventing of methemoglobinemia, 2) increasing shelf life of juice or other foods products, 3) decreasing blood pressure in a subject, 4) treating ischemia in a subject, 5) preventing coagulation of platelets either in blood or other blood products such as platelet rich plasma (PRP), 6) increasing athletic performance in a subject, 7) increasing muscle strength (as evidenced by amounts of repetitions and/or total weight a subject can lift) in a subject, 8) increasing endurance (as measured by VO₂ₘₐₓ and/or time to exhaustion) in a subject, 9) decreasing lactic acid concentration present in muscles after exercise (as measured by blood lactate levels before during and after exercise) in a subject, 10) treating low oxygen saturation/hypoxia in a subject, 11) treating gastrointestinal ulcers in a subject, 12) treating a viral, fungal, bacterial, protozoa, or parasitical infection in a subject, 13) treating migraine in a subject, 14) treating hypertension in a subject, 15) administering blood transfusions to a subject, 16) keeping stable blood flow of a subject during surgeries (for example cardiovascular surgeries), 17) treating diabetes in a subject, treating metabolic dysfunction in a subject, 18) healing a wound in the subject, 19) treating erectile dysfunction in a subject, 20) treating alopecia in a subject, 21) increasing SpO₂ in a subject (for example, where the subject exhibits signs of hypoxia, such has having SpO₂ of less than 95%), 22) reducing symptoms of diseases that cause reduction in SpO₂ in a subject; and 23) reducing inflammation and/or signs, symptoms, and/or markers of inflammation in a subject, including, but not limited to, reductions of or resulting in reductions of F2-isoprostane. The methods all involve circumstances where administration of NO and/or H₂ would be beneficial. In some implementations, the methods comprise administering the NOH2 solution described herein to the subject. In other implementations, the methods comprise infusing the liquid in question (e.g., juice, liquid food product, blood or plasma) with NO and H₂ according to the methods described herein.

The disclosed compositions, such as NOH2 water for example, are useful in any application where H₂ water may be used, such as providing an antioxidant source, providing antiaging benefits, improving recovery from exercise, reducing inflammation, reducing free radicals in the body, improving mood, and reducing cholesterol.

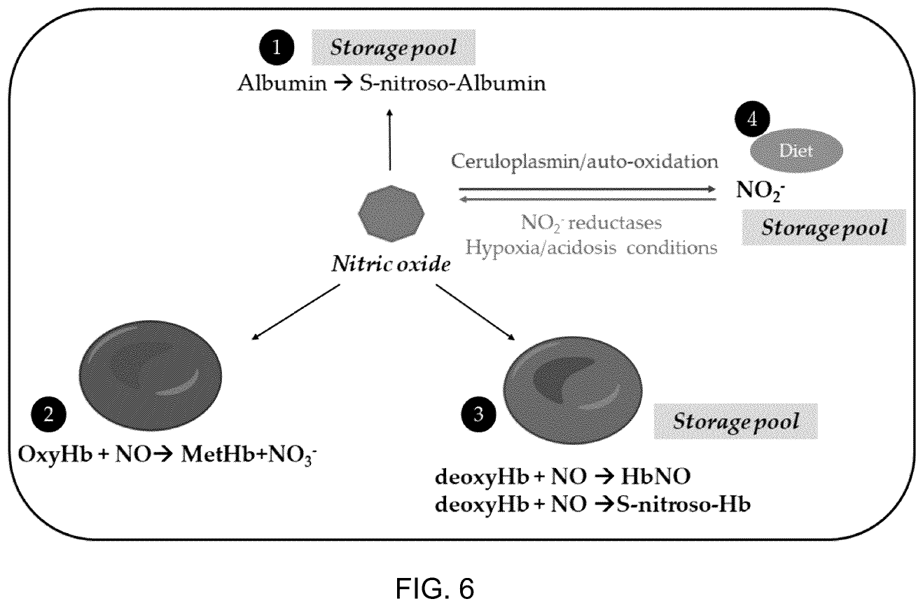

With respect to the method of treating and/or preventing methemoglobinemia, co-administration of NO and H₂ prevents the onset of methemoglobinemia or reduces the likelihood of developing methemoglobinemia from NO therapy. This is an important solution to a well-known drawback of using NO to treat a disease or condition or to enhance athletic performance, as administration of NO is known to induce methemoglobinemia, as illustrated in FIG. 6. It is in part because of the risk of developing methemoglobinemia that administration of an NO-infused solution is considered a failed therapeutic strategy for the treatment of pulmonary hypertension. It should be noted also that NO+H₂ solutions increased oxygenation during exercise which is highly surprising since in a study measuring the effects of NO inhalation in oxygenation during exercise “inhaled NO worsened the hypoxaemia at all exercise levels” (Durand et al. “Effects of nitric oxide inhalation on pulmonary gas exchange during exercise in highly trained athletes”; attached as an appendix to this application).

Methemoglobin formation can be detected in vivo by drops in oxygen saturation that can be conventionally measured by a pulse oximeter. Alternatively, it can be detected by changes in blood color compared to control blood or observing changes in blood color upon the passing of oxygen. For example, blood samples with a methemoglobin concentration of >20% have an evident chocolate-brown color. To distinguish this, a person may pass 100% oxygen in a tube that contains the dark blood. If the blood continues to remain chocolate-brown/dark, it indicates the presence of methemoglobin. An alternative is to place one to two drops of blood on a white filter paper and expose it to atmospheric oxygen. If the chocolate-brown color does not change with time, then the initial color of the blood is due to the presence of methemoglobin. This alternative test can be accelerated by gently blowing supplemental oxygen onto the filter paper. Finally methemoglobinemia can be detected by UV-Vis spectrometry of lysed blood or through a co-oximeter.

Exemplary methods of administration of the solutions are either oral (such as ingestion, or tube feeding), inhaled such as with a nebulizer or atomizer, infusion to the blood, or topical application. However other routes and forms of administration could be possible utilizing the methods and compositions described herein, including immediate release tablets or capsules (where the excipient disintegrates almost immediately in contact with the saliva or gastric fluid), delayed release tablets or capsules and the like, sustained release formulations (where one or more of the constituents of the formulation are released on the gastric tract over a long period of time), sublingual tablets, direct administration of the constituents via a catheter or feeding tube to the G.I. tract, liquid caps, buccal delivery, injection to the body through possible routes such as intravenous, intramuscular, subcutaneous, intraosseous, intrathecal and intradermal and the like.

Also disclosed herein are methods of increasing NO levels in a subject or of increasing plasma H₂ level in a subject. The methods may comprise orally administering to the subject the NOH2 solution (such as NOH2 water) described herein. In one aspect, the methods comprise orally administering to the to the subject at least 100 ml of a composition prepared according to a method comprising: combining 10 g KNO₃, 2.5 g magnesium metal powder (60-200 mesh size), and 19 g of citric acid to produce a gaseous composition comprising NO and H₂ and diffusing 500 ml water with the produced gaseous composition at 0° C. for 5 minutes. In another aspect, the composition is prepared by diffusing through water or other suitable liquid H₂ and NO from gas tank(s).

The contents of all references, patents, and published patent applications cited throughout this specification, if any, are incorporated herein by reference in their entirety for all purposes.

### EXAMPLES

The present disclosure is further illustrated by the following examples that should not be construed as limiting.

### Example 1

NO in combination with H₂ were conveniently produced by mixing 10 g KNO₃ with 2.5 g magnesium metal powder (60-200 mesh size) and 19 grams of citric acid in a 250 ml Kipp's hydrogen generator. A plastic tube connected the gas exhaust to a 500 ml plastic water bottle containing 500 ml of water at 0° C. The gas mixture was left to diffuse in the bottle for 5 minutes. Immediately afterwards the water was transferred to a 1000 ml beaker. NO and H₂ concentration in the NOH2 water was measured indirectly by placing the beaker in a AT09p7 Vacuum Oven (0.8 cubic feet). H₂ and NO concentrations in the chamber were measured at 196 ppm and 18 ppm respectively. Based on calculations, infusing water with the gases produced by combining potassium nitrate, magnesium metal, and citric acid resulted in 0.7 mg of NO dissolved in 500 ml of water, (Other measurements of gas concentrations in other examples herein were collected in the chamber and reported accordingly in a similar manner here, unless otherwise indicated.)

### Example 2

To measure the shelf life of the NOH2 water upon exposure to air, water was prepared as per Example 1 and was left open at room temperature for 3.5 hours. Using the same methodology as Example 1, it was found that the water still contained nitric oxide and hydrogen (12 ppm and 108 ppm chamber readings, respectively). Thus, despite the art teaching that NO survives mere minutes in water upon exposure to air, NOH2 water prepared per Example 1 remained stable for hours.

### Example 3

To measure the stability of NO and H₂ concentration in the NOH2 water in atmospheric conditions and sunlight, 500 ml of NOH2 water was prepared as per Example 1 with H₂ and NO chamber concentrations of 196 ppm and 18 ppm, respectively, and was stored in a sealed clear mason jar and left outdoors in direct sunlight for 3 months. After three months, the concentration of NO and H₂ after the mason jar was unsealed was detected to be 17 ppm for NO and 168 ppm for H₂

### Example 4

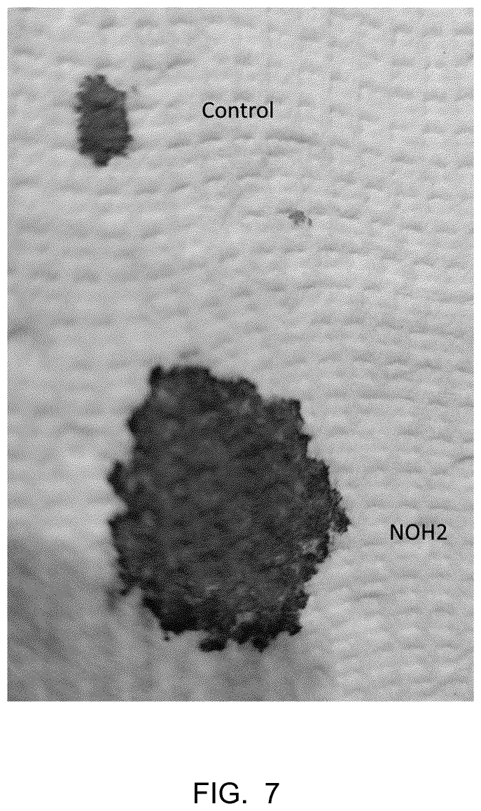

To test if the combination of NO and H₂ causes methemoglobinemia, the gas produced as described in Example 1 was diffused in heparinized bovine blood at 0° C. as shown in FIG. 7. No change of the color of the blood took place, indicating that no methemoglobin was formed.

### Example 5

The NOH2 blood of Example 4 was put into the vacuum oven with NO and H₂ sensors. Upon warming up the blood to 60° C., the concentration of NO in the chamber rose to 13.8 ppm. Thus, despite the vast amount of prior art teaching that NO would be unstable and rapidly disappear in blood, the disclosed method managed to infuse blood with NO and enabled blood to retain the gas for hours. Surprisingly, H₂ rose even more than in the case of water, up to 552 ppm.

### Example 6

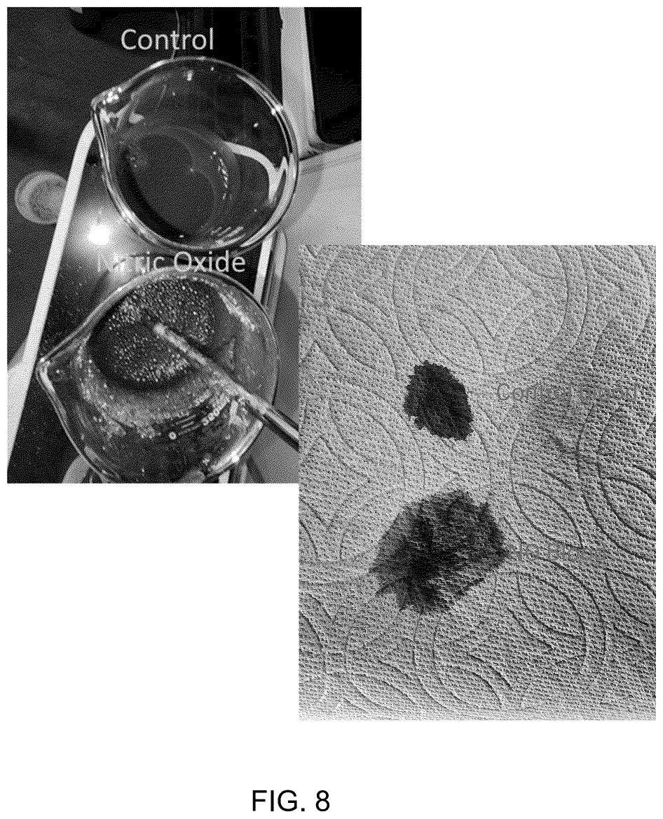

NO (40 ppm) was diffused in blood (without diffusing H₂). The blood changed color to a dark red, characteristic of methemoglobinemia as shown in FIG. 8.

### Example 7

To test the ability of juices to hold NO and H₂, beet juice was infused with the gas mixture of Example 1 and was left in fridge at about 40° F. (4° C.) for 3 days. Beet juice was heated to 60° C. for measurement of NO and H₂ concentrations. The sensors in the chamber registered 11.3 ppm for NO and 857 ppm for H₂.

### Example 8

NOH2 beet juice was infused with the gas mixture of Example 1 was put in a jar and shaken vigorously to increase contact with atmospheric air. After which, NO and H₂ concentrations in the NOH2 beet juice were measured and the sensors registered 7.6 ppm for NO and 73 ppm for H₂.

### Example 9

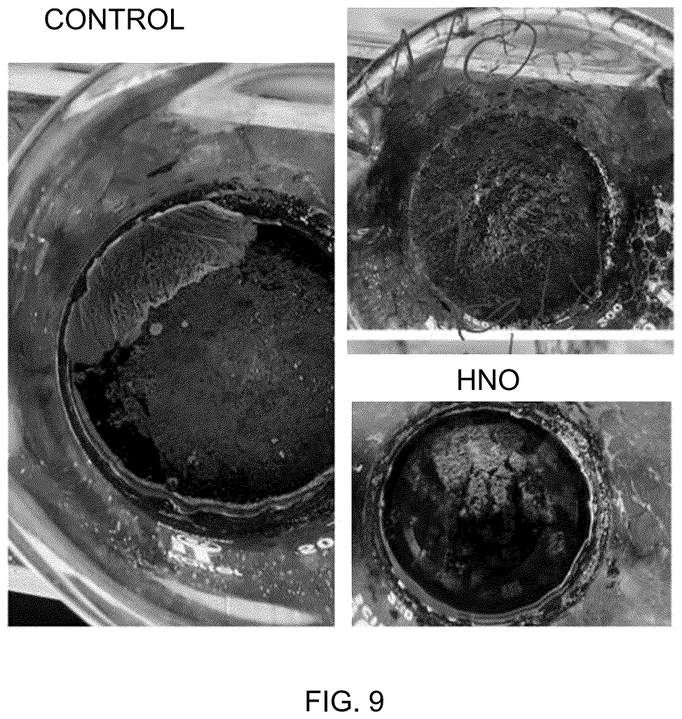

To test the ability of NO and H₂ to retard spoilage, a first sample of beet juice was diffused with NO only (“untreated beet juice”) and a second sample of beet juice was diffused with the combination of NO and H₂ through the gas mixture of Example 1 (“treated beet juice”). The treated beet juice (“NOH2 beet juice”) and the untreated beet juice were both left out in the open air for one week. The NOH2 beet juice visually showed significantly less spoilage than the untreated beet juice as shown in FIG. 9.

The experiment was repeated with juices from pear, apple, and orange. All of them exhibited significantly less spoilage after treatment with NOH2. Other tests to confirm these results would include measuring the total amount of grown fungi and measuring the surface area of the fungi grown.

### Example 10

To see if administration of H₂ can reverse methemoglobinemia, methemoglobin formation was first induced by diffusing blood in a vial with 40 ppm NO (and no H₂). H₂ and O₂ (99.99% purity or higher) were then diffused in the blood containing methemoglobin. The blood regained its red color.

### Example 11

Bronchoconstriction was caused on a subject by having the subject inhale a nebulized pilocarpine solution (25 mg/L in 0.9% saline). Upon inhaling the subject could feel the onset of bronchoconstriction indicated by difficulty in breathing. Upon inhaling nebulized NOH2 water, his breathing became normal, indicating a bronchodilating effect of NOH2 water.

### Example 12

NOH2 water prepared as per Example 1 was measured for oxygen content using a RCYAGO Dissolved Oxygen Meter. The water was found to contain 7.2 mg/L dissolved oxygen.

Thus, surprisingly, in the presence of oxygen, NO remained stable in spite of the increased concentration of NO and H₂. The pH of the NOH2 water was surprisingly found to be alkaline (above 7) despite the known phenomenon that NO₂ would form HNO₃, which would make it acidic.

### Example 13

A 43-year-old recreationally active male used the treadmill at 5 miles per hour. His time to exhaustion was 14 minutes. After a week without further training, he imbibed 500 ml of NOH2 water 10 minutes prior to repeating the same exercise. His time to exhaustion was increased to 17 minutes. After a one-week washout the subject repeated the treadmill exercise without prior ingestion of NOH2 water and measured his lactate levels using a Heartscare C1 lactate monitoring system. His blood lactate measured at 7.9 mmol/L. After another one-week washout, he repeated the treadmill exercise 30 minutes after imbibing 500 ml of NOH2 water. His blood lactate measured at 6.3 mmol/L.

### Example 14

A 43-year-old recreationally active male bench pressed 100 kg (220 lbs) for 6 repetitions. After a week without further training, he imbibed 500 ml of NOH2 water 10 minutes prior to repeating the same exercise. He managed to perform 9 repetitions.

### Example 15

Blood was collected from a 43-year-old male subject (weighing 122 kg (270 lbs)) and stored into a tube with no anticoagulants on two occasions. During each occasion, three vials of 80 ml blood each were collected. The first occasion was after 16 hours of overnight fasting where he was only allowed to drink water. The second occasion was 40 minutes after the male subject drank 1000 ml of NOH2 water prepared fresh as per example 1 in 0° C. temperature water.

The blood samples were centrifuged on a Drucker 614 B centrifuge for 30 minutes. Plasma (˜30 ml) was clearly formed in the top which was used to take samples for the experiments. Using the supernatant plasma, the following related experiments were conducted.

### UV-Vis Spectra of Collected Plasma:

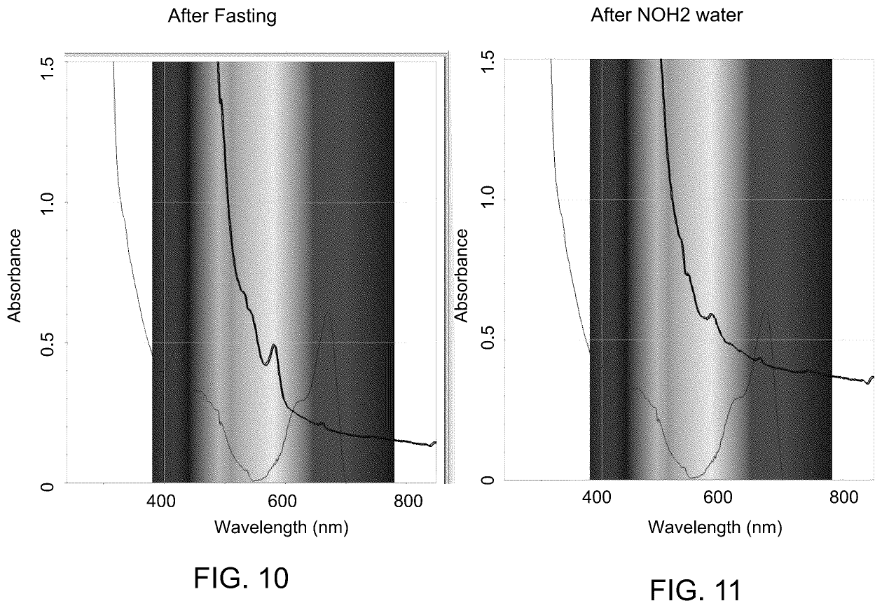

A 5-ml cuvette containing 5 ml of plasma was placed into a Vernier UV-Vis spectrophotometer to produce an ultraviolet-visible (“UV-Vis”) spectra of the plasma after fasting and after NOH2 water consumption. The UV-Vis spectra before NOH2 water consumption (FIG. 10) and after NOH2 water consumption (FIG. 11), indicates that the NOH2 water has biological activity and caused changes in the plasma spectra.

Changes in NO Level in the Blood Stream from NOH2 Water:

To see if any NO from the NOH2 water increases NO level in the blood stream, ABTS (2,2′-azinobis(3-ethylbenzothiazoline-6-sulfonic acid) was used as an indicator. A solution of 50 mg of ABTS in distilled water in 100 ml water was prepared. The solution displayed a blue color, with λmax around ˜420 nm, matching to what was described in the literature. It has been described that reaction of ABTS with NO diminishes this absorption. The ABTS solution (10 ml) was combined with 4 ml of the collected plasma samples. 5 ml of this solution was used for spectra measurement.

Despite the knowledge in the field suggesting the drinking water comprising NO would not change NO levels detectible in plasma (such as due to its half-life in blood of scant milliseconds), a comparison of the plasma spectra showed a profound decrease of absorption at 420 nm, which is the wavelength indicating presence of NO.

H₂ Retention from NOH2 Water:

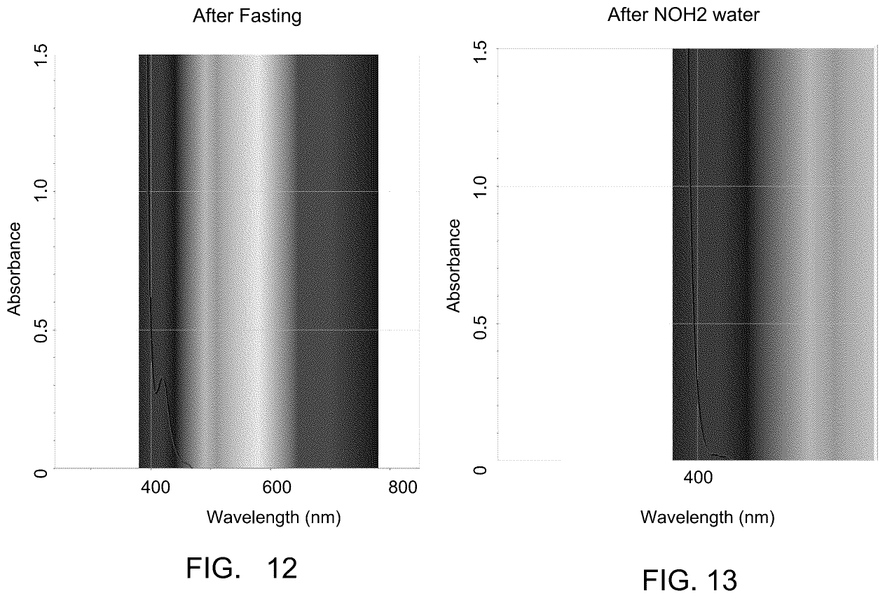

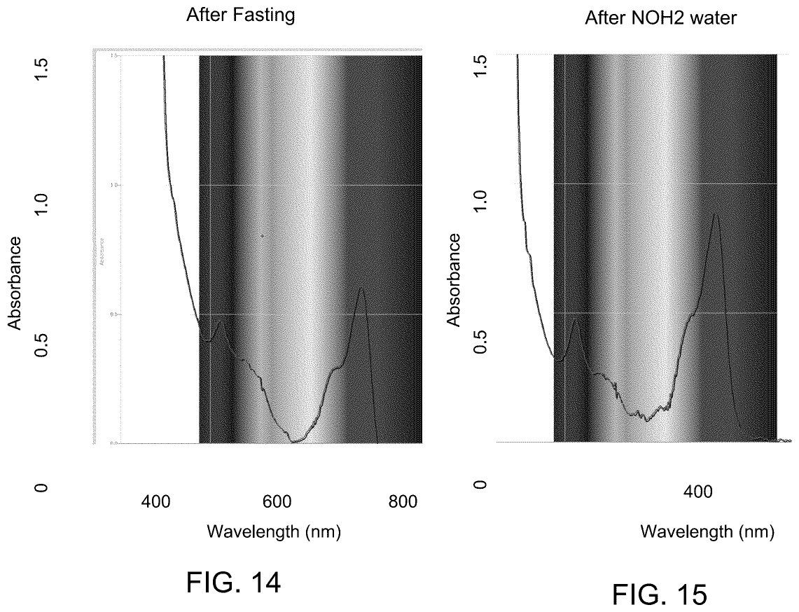

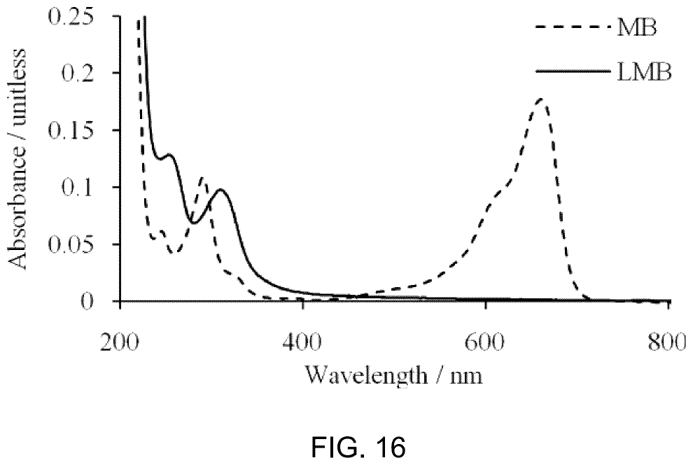

To measure retention of H₂ from the ingestion of 1000 ml NOH2 water, one drop of Kueysing Hydrogen Test Reagent was added in 10 ml of the collected plasma. The method relies on the reduction of methylene blue (MB) to leucomethylene blue (LMB) by hydrogen reduction as shown in FIG. 16. A good indicator at smaller concentrations is diminishing of absorption at ˜660 nm. As shown in FIGS. 12 and 13, the UV-Vis spectra for the plasma taken after ingestion of NOH2 water (right) shows a much more profound absorption at ˜660 nm, implying a greater conversion of methylene blue to leucomethylene blue due to reaction with H₂. This also means a greater in vivo antioxidant (reducing) capability of NOH2 water compared to hydrogen water. It should also be noted that surprisingly, consumption of 1000 ml of regular H₂ water (Elevate-brand hydrogen water), showed no change in the UV-Vis spectra at 660 nm using the same measurement conditions. See FIGS. 14 and 15.

### Example 16

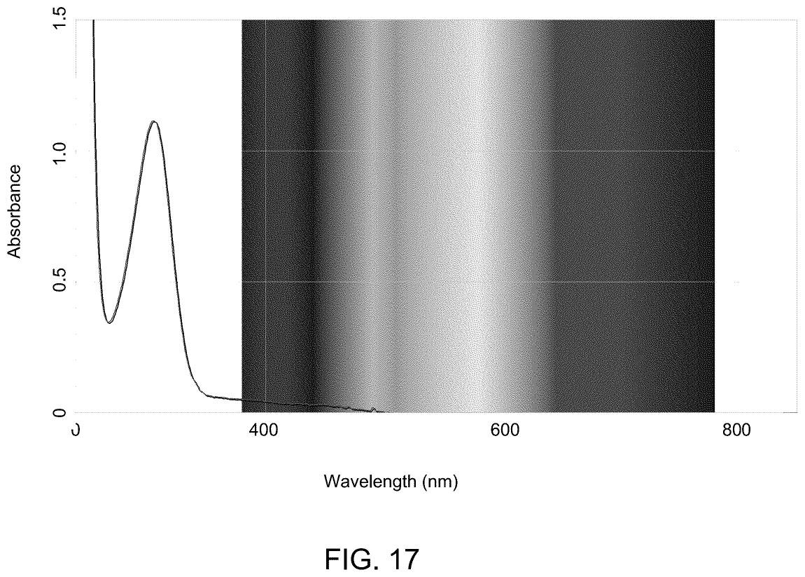

A UV-Vis spectra of freshly prepared (less than 10 minutes after diffusion of the gas) NOH2 water was taken and shown in FIG. 17.

### Example 17

F2-isoprostane is a marker for measuring inflation. F2-isoprostane levels are generally higher in smokers compared to non-smokers as a result of the oxidative damage caused by cigarette smoke. (e.g., oxidative damage to proteins, DNA, and lipids, contributing to the pathobiology of atherosclerosis, heart disease, and cancer). A reduction in F2-isoprostane is a sign of a reduction in inflammation and oxidative stress.

A 44-year-old non-smoker male smoked six Marlboro Red cigarettes over a four-hour period and experienced an increase of F2-isoprostane levels (a major marker of inflammation) approximately 44%. A few days later, subject repeated the process while drinking 80 oz of NOH2 water (prepared as the water was in Example 1) and experienced a decrease of F2-isoprostane levels of approximately 24%. This was an astonishingly unexpected result because supplementation with regular hydrogen has been reported to purportedly decrease isoprostanes by just single-digit numbers.

### Example 18

A 42-year-old female smoker (1+ packs per day Marlboro Reds for 10+ years) weighing approximately 50 kg (approximately 110 lbs) had baseline F2-isoprostane levels of 1.31 ng/ml. Without changing any other daily or dietary habits or exercise, subject consumed 64 oz of NOH2 water (prepared as the water was in Example 1) per day for two days consecutively. In the morning of the 3rd day, subject's F2-isoprostane levels were 0.20 ng/ml (a roughly 85% reduction).

1. A nitric oxide stabilized liquid composition comprising:
a liquid;
nitric oxide gas (NO); and
hydrogen gas (H₂);
wherein the NO and the H₂ are diffused in the liquid to produce the nitric oxide stabilized liquid.

2. The composition of claim 1, wherein the NO and the H₂ are diffused in the liquid comprises the NO and the H₂ being dissolved in the liquid.

3. The composition of claim 1, wherein the liquid comprises water.

4. The composition of claim 1, wherein concentration of the NO and the H₂ are respectively at least 1 ppm and at least 100 ppb.

5. The composition of claim 1 further comprising a pharmaceutically acceptable additive.

6. The composition of claim 5, wherein the pharmaceutically acceptable additive is selected from: a carrier, an excipient, a binder, a colorant, a flavoring agent, a preservative, a buffer, a diluent, and combinations thereof.

7. The composition of claim 5, wherein the pharmaceutically acceptable additive is a food additive.

8. The composition of claim 1, wherein the composition is in an administration container.

9. A nitric oxide and hydrogen (NOH2) administration container comprising:
a) a liquid;
b) nitric oxide gas (NO); and
c) hydrogen gas (H₂),
d) wherein the NO and the H₂ are diffused in the liquid.

10. The NOH2 administration container of claim 9, wherein the NO and the H₂ are dissolved in the liquid.

11. The NOH2 administration container of claim 9, wherein the liquid comprises water.

12. The NOH2 administration container of claim 9, wherein concentration of the NO and the H₂ are respectively at least 1 ppm and at least 100 ppb.

13. The NOH2 administration container of claim 9, further comprising a pharmaceutically acceptable additive.

14. The NOH2 administration container of claim 13, wherein the pharmaceutically acceptable additive is selected from the group consisting of: a carrier, an excipient, a binder, a colorant, a flavoring agent, a preservative, a buffer, a diluent, and combinations thereof.

15. The NOH2 administration container of claim 13, wherein the pharmaceutically acceptable additive is a botanical additive derived from a plant including a fruit of a plant.

16. The NOH2 administration container of claim 13, wherein the liquid comprises a botanical liquid.

17. The NOH2 administration container of claim 16, wherein the botanical liquid is a fruit juice.

18. The NOH2 administration container of claim 13, wherein the pharmaceutically acceptable additive is a food additive.

19. The NOH2 administration container of claim 18, wherein the food additive is a liquid.

20. The NOH2 administration container of claim 19, wherein the liquid is milk.

21. The NOH2 administration container of claim 9, wherein the liquid comprises blood.

22. The NOH2 administration container of claim 9, wherein the administration container is a beverage container comprising a container wall with a consumption opening and an opening closure that seals the nitric oxide stabilized liquid in the container.

23. The NOH2 administration container of claim 22, wherein the beverage container is pressurized with NO.

24. The NOH2 administration container of claim 22, wherein the beverage container is a metal can.

25. The NOH2 administration container of claim 24, wherein the beverage container is an aluminum can.

26. The NOH2 administration container of claim 22, wherein the beverage container is pressurized to 2 atmospheres or more.

27. The NOH2 administration container of claim 26, wherein the beverage container is pressurized to 4 atmospheres or more.

28. The NOH2 administration container of claim 27, wherein the beverage container is pressurized to 6 atmospheres or more.

29. The NOH2 administration container of claim 9, wherein the administration container is an intravenous (IV) container.

30. The NOH2 administration container of claim 9, wherein the administration container is a vape container configured for insertion into a vape device for inhalation of the nitric oxide stabilized liquid as a vapor therefrom.

31. The NOH2 administration container of claim 9, wherein the administration container is capsule for oral consumption.

32. A method of producing a nitric oxide stabilized liquid having stabilized nitric oxide gas (NO) dissolved therein comprising:
a) providing a nitric oxide liquid with NO dissolved therein with a concentration of least 1 ppm;
b) providing hydrogen gas (H₂); and
c) dissolving the H₂ in the liquid at a concentration of at least 100 ppb to produce the nitric oxide stabilized liquid.

33. The method of claim 32, wherein the nitric oxide stabilized liquid comprises oxygen.

34. The method of claim 32, wherein the NO concentration in the liquid is stable having less than a 25% drop in NO concentration in 24 hours after the addition of H₂.

35. The method of claim 34, wherein providing a nitric oxide liquid with NO dissolved therein with a concentration of least 1 ppm comprises:
a) providing NO;
b) providing a liquid; and
c) dissolving the NO in the liquid to produce the nitric oxide liquid with a concentration of least 1 ppm.

36. A method of producing a nitric oxide stabilized liquid comprising:
a) providing a liquid;
b) providing nitric oxide gas (NO);
c) providing hydrogen gas (H₂); and
d) simultaneously dissolving in the liquid the NO and the H₂ to produce the nitric oxide stabilized liquid.

37. The method of claim 36, wherein the NO is at a concentration of least 1 ppm and the H₂ is at a concentration of at least 100 ppb in the nitric oxide stabilized liquid.

38. The method of claim 36, wherein the nitric oxide stabilized liquid comprises oxygen.

39. The method of claim 36, wherein the NO concentration in the liquid is stable having less than a 25% drop in NO concentration in 24 hours after the simultaneous addition of NO and H₂.

40. A method of increasing nitric oxide levels in blood of a subject comprising:
a) providing a nitric oxide stabilized liquid comprising nitric oxide gas (NO) at a concentration of at least 1 ppm and hydrogen gas (H₂) at a concentration of least 100 ppb in the nitric oxide stabilized liquid;
b) administering the nitric oxide stabilized liquid to the subject.

41. The method of claim 40, wherein administering the nitric oxide stabilized liquid includes drinking the nitric oxide stabilized liquid.

42. The method of claim 41, wherein the nitric oxide stabilized liquid is administered in a dose of 1-10,000 ml.

43. The method of claim 42, wherein the nitric oxide stabilized liquid is administered in a dose of 10-5,000 ml.

44. The method of claim 43, wherein the nitric oxide stabilized liquid is administered in a dose of 100-1,000 ml.

45. The method of claim 40, wherein administering the nitric oxide stabilized liquid includes intravenous introduction of the nitric oxide stabilized liquid.

46. The method of claim 45, wherein the nitric oxide stabilized liquid is administered at an infusion rate of 0.1-10 ml/min.

47. The method of claim 40, wherein the method is for treating methemoglobinemia.

48. The method of claim 40, wherein the method is for treating any one of: hypertension; atherosclerosis; varicose veins; low oxygen saturation/hypoxia; gastrointestinal ulcers; viral, fungal, bacterial, protozoa, or parasitic infections; migraine; or anemia.

49. The method of claim 40, wherein the method is for reducing inflammation.

50. The method of claim 40, wherein the method is for reducing F2-isoprostane.
